(19) United States
(12) Patent Application Publication
(10) Pub. No.: US 2010/0268317 A1
(43) Pub. Date: Oct. 21, 2010

(54) Stent Graft Restraining Mechanism for a Delivery System (Catheter)

(75) Inventors: Mark Stiger, Windsor, CA (US); Jia-Hua Xiao, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/426,011

(22) Filed: Apr. 17, 2009

(51) Int. Cl. A61F 2/84

(52) U.S. Cl. 623/1.12

(57) ABSTRACT

A prosthesis delivery system comprises a tip having a lumen, a first end and a second end; a sleeve coupled to the tip second end; a spindle comprising a spindle body and a plurality of projections extending radially outward from the spindle body, each projection having a first edge and a second edge, the first edge facing the tip and the second edge extending from the first edge, being angled toward the tip first end, and extending toward the sleeve; and endoprosthesis having an undulating spring with a plurality of crown portions having apices, where the crown portions extend over at least one of the first or second edges and the sleeve radially constrains the apices.

### FIELD OF THE INVENTION

The invention relates to grafts suitable for placement in a human body lumen such as an artery.

### BACKGROUND OF THE INVENTION

Tubular prostheses such as stents, grafts, and stent-grafts (e.g., stents having an inner and/or outer covering comprising graft material and which may be referred to as covered stents) have been used to treat abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., polyester material such as Dacron® fabric, polytetrafluoroethylene PTFE, or expanded polytetrafluoroethylene (ePTFE) or some other polymer) supported by a framework (e.g., one or more stent or stent-like structures) to treat or isolate aneurysms. The framework provides mechanical support and the graft material or liner provides a blood barrier. Approaches for making stent-grafts have included sewing one or more stents or annular metallic spring elements, which may have a sinusoidal configuration, to woven materials, ePTFE, PTFE or Dacron® fabric. Other approaches have included electrospinning the stent structure with a polymer or dip coating. Many stent-grafts have a bare-spring or crown stent attached to one or both of its ends to enhance fixation between the stent-graft and the vessel where it is deployed. The bare-spring or crown stent can be referred to as an anchoring device. In treating an aneurysm, the graft material typically forms a blood impervious lumen to facilitate endovascular exclusion of the aneurysm.

In treating an aneurysm, the graft material typically forms a blood impervious lumen to facilitate endovascular exclusion of the aneurysm. When using a stent-graft, the stent-graft typically is placed so that one end of the stent-graft is situated proximal to or upstream of the diseased portion of the vessel and the other end of the stent-graft is situated distal to or downstream of the diseased portion of the vessel. In this manner, the stent-graft extends through and spans the aneurysmal sac and extends beyond the proximal and distal ends thereof to replace or bypass the dilated wall. The graft material typically forms a blood impervious wall with a lumen therein to facilitate endovascular exclusion of the aneurysm.

Such prostheses can be implanted in an open surgical procedure or with a minimally invasive endovascular approach. Minimally invasive endovascular stent-graft use is preferred by many physicians over traditional open surgery techniques where the diseased vessel is surgically opened, and a graft is sutured into position bypassing the aneurysm. The endovascular approach, which has been used to deliver stents, grafts, and stent-grafts, generally involves cutting through the skin to access a lumen of the vasculature. Alternatively, vascular access may be achieved percutaneously via successive dilation at a less traumatic entry point. Once access is achieved, the stent-graft can be routed through the vasculature to the target site where it is deployed.

When using a balloon expandable stent-graft, balloon catheters generally are used to expand the stent-graft after it is positioned at the target site. When, however, a self-expanding stent-graft is used, the stent-graft generally is radially compressed or folded and placed at the distal end of a sheath or delivery catheter and self expands upon retraction or removal of the sheath at the target site. More specifically, a delivery catheter having coaxial inner and outer tubes arranged for relative axial movement can be used. The stent-graft is compressed and disposed within the distal end of an outer catheter tube in front of an inner tube. A delivery catheter is typically routed though a vessel, until the end of the catheter (and the stent-graft) is positioned in the vicinity of the intended treatment site. The inner tube is then held stationary while the outer tube of the delivery catheter is withdrawn. The inner tube or stop prevents the stent-graft from moving back as the outer tube is withdrawn. As the outer tube is withdrawn, the stent-graft is gradually exposed from a proximal end to a distal end of the stent-graft, the exposed portion of the stent-graft radially expands so that at least a portion of the expanded portion is in substantially conforming surface contact with a portion of the blood vessel wall. The proximal end of the stent-graft is the end closest to the heart by way of blood flow path whereas the distal end is the end away from the heart during deployment. In contrast and of note, the distal end of the catheter is usually identified to the end that is farthest from the operator while the proximal end of the catheter is the end nearest the operator. Depending on the access location the stent graft and delivery system description may be consistent or opposite. An exemplary stent-graft delivery system is described in U.S. Pat. No. 7,264,632 to Wright et al., the disclosure of which is hereby incorporated herein in its entirety by reference thereto.

There remains a need to improve endolumenal prosthesis delivery control and placement.

### SUMMARY OF THE INVENTION

The present invention involves improvements in prostheses delivery systems.

In one embodiment according to the invention, a prosthesis delivery system comprises a tip having a lumen, a first end and a second end; a sleeve coupled to the tip second end; a spindle comprising a spindle body and a plurality of projections extending radially outward from the spindle body, each projection having a first edge and a second edge, the first edge facing the tip and the second edge extending from the first edge, being angled toward the tip first end, and extending toward the sleeve; and endoprosthesis having an undulating spring with a plurality of crown portions having apices, where the crown portions extend over at least one of the first or second edges and the sleeve radially constrains the crowns (apices).

### BRIEF DESCRIPTION OF THE DRAWINGS

FIG. 1 is a partial cross-sectional view of a stent-graft delivery system without a stent-graft and outer sheath according to one embodiment of the invention.

FIG. 2 is a partial cross-sectional view of the stent-graft delivery system of FIG. 1 including a stent-graft located within a retractable primary sheath in a pre-deployment un-retracted position.

FIG. 3 is a partial cross-sectional view of the stent-graft delivery system of FIG. 2 with the retractable primary sheath partially retracted.

FIG. 4 is a partial cross-sectional view of the stent-graft delivery system of FIG. 3 after deployment of a proximal anchor stent ring of the stent-graft.

FIG. 5 is a perspective view of an expanded stent-graft similar to the stent-graft of FIGS. 2, 3 and 4.

FIG. 6 is an enlarged partial longitudinal schematic sectional view of a distal section of the delivery system of FIGS. 1-4.

FIGS. 6A-E schematically illustrate the progressive steps of a method of deploying the stent graft from the distal section shown in FIG. 6 to place the stent-graft proximal graft edge below the ostia of branch vessels, where FIG. 6A shows a stent-graft loaded in the stent-graft delivery system, FIG. 6B shows partial retraction of the stent-graft primary sheath, FIG. 6C shows the proximal crowns of the stent-graft proximal spring pivoted outward to allow controlled full deployment of the proximal edge of the stent-graft graft so that the graft engages the vessel wall prior to release of the proximal crowns of the proximal spring, FIG. 6D shows advancement of the distal tip sleeve constraint to release the proximal crown of the proximal spring, and FIG. 6E shows the stent-graft fully deployed and the delivery apparatus removed.

FIG. 7 schematically illustrates deployment of a stent-graft in a curved vessel using the delivery system of FIG. 1.

FIG. 8 is a schematic diagram illustrating a stent-graft deployment delivery system incorporating the apparatus of FIG. 1

### DETAILED DESCRIPTION

The following description will be made with reference to the drawings where when referring to the various figures, it should be understood that like numerals or characters indicate like elements.

In one embodiment according to the invention, a self-expanding prosthesis deployment apparatus comprises a spindle and a prosthesis proximal spring proximal crown constraining sleeve surrounding a portion of the spindle where the spindle is configured to allow crowns of the proximal spring to pivot when a distal portion of the proximal spring is expanded and spaced from the proximal crown of the proximal spring. With this configuration, the amount of sleeve displacement needed to release the crowns of the proximal spring can be minimized. Limiting the amount of sleeve displacement has many benefits. Among the benefits of limiting the amount of sleeve displacement are minimizing the corresponding space needed by the tip release mechanism at the handle. In one embodiment, once the system is ready to deploy, only 2 mm of movement is required to release the proximal spring apices (crowns). A reduced amount of required component separation (separation of the sleeve from the spindle) also limits the propensity of the system to catch on the deployed prosthesis (e.g., stent-graft). When the system catches on the deployed prosthesis, deployment complications arise, which can induce trauma to adjacent anatomy. Further, closing the mechanism after deployment is also limited to the 2 mm of deployment movement. The pivot enabling configuration which provides a predictable and circumferentially uniform deployment can also enhance the ability to maintain the prosthesis centered in the vessel during deployment, which can improve deployment accuracy. Other features, advantages, and embodiments will be apparent to those skilled in the art from the following description and accompanying drawings.

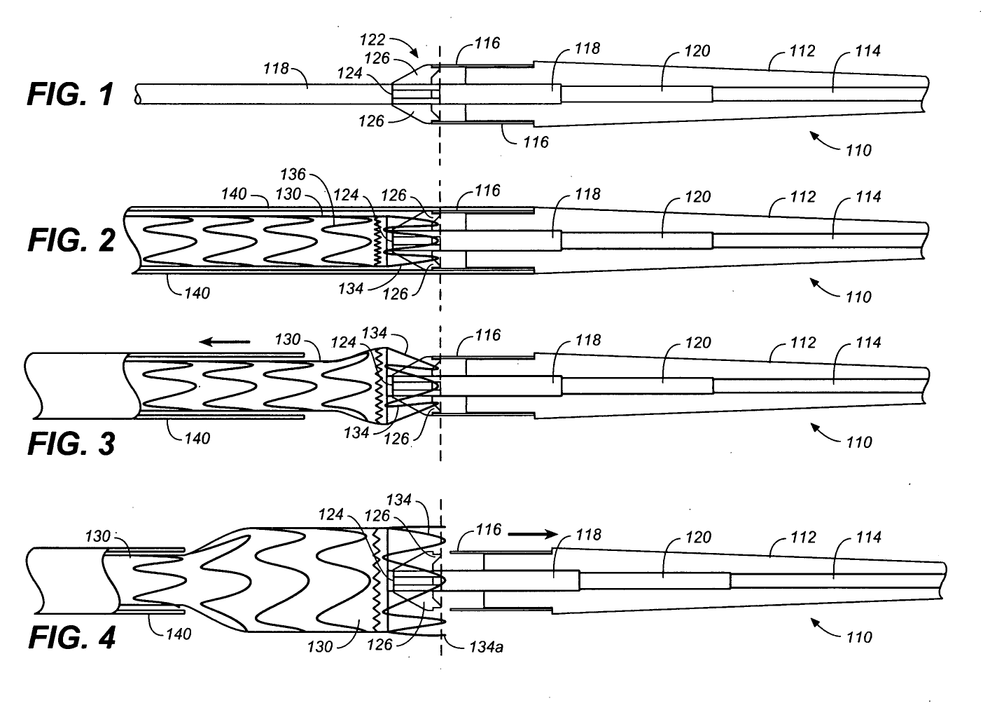

FIG. 1 illustrates the distal tapered tip portion of the delivery system 110 alone without a stent-graft, while FIGS. 2-4 show close up views of the deployment delivery system tip portion loaded with a stent-graft 130, with the figures progressively showing deployment from within a retractable primary sheath 140. This system could also deploy a stent alone or some other form of endoprosthesis.

A configuration of the stent-graft deployment system 110 includes a tapered tip 112 that is flexible and able to provide trackability in tight and tortuous vessels. The tapered tip 112 can include a lumen 114 formed therein to allow for passage of a guidewire for example. Other tip shapes such as bullet-shaped tips could also be used.

Referring to FIG. 2, retractable primary sheath 140 (preferably made of a semi-rigid material such as PTFE) is in an un-retracted position with stent-graft 130 disposed therein. Sheath 140 retrains stent-graft 130 in a first constrained diameter configuration. Outer tube 118 is located in the retractable primary sheath 140 and in the stent-graft 130. Inner tube 120 is slidably disposed in outer tube 118 and serves as a guidewire lumen. Inner tube 120 and outer tube 118 can move along the longitudinal axis relative to each other and can also move along the longitudinal axis relative to retractable primary sheath 140. Tubular sleeve 116, which is coupled to distal tapered tip 112 or can be integrally formed therewith, is configured to radially constrain at least a portion of a proximal end of stent-graft 130 in a radially compressed configuration. Actuating members at the operator's end of the catheter create a forces in an axial direction to provide a controlled relative axial movement between the outer tube 118 and inner tube 120 to precisely control the release of the proximal end of the stent-graft (e.g., the proximal spring apices (crowns)) as will be described in more detail below.

Referring to FIG. 3, sheath 140 is partially retracted. The proximal end (tip) of the stent-graft 130 remains constrained, while a proximal portion of the stent-graft 130 (that is now exposed due to the partial retraction of the sheath 140) between the end of the sheath 140 and the constrained proximal end (tip) is partially expanded deployed. This allows longitudinal repositioning of the stent-graft before releasing the proximal end (the release of the proximal end of the stent-graft prevents repositioning of the stent-graft in a direction toward the proximal end of the stent-graft, while depending on the degree of expansion and contact between the stent-graft and the wall of the vessel in which the stent graft is being deployed, some pull down (movement toward the distal end of the stent graft) of the stent graft is possible.

Referring to FIGS. 1 and 4, stent delivery system 100 includes stent holding spindle 122. Spindle 122 includes a tubular spindle body 124, which surrounds and is secured to outer tube 118, and a plurality of radially extending projections 126a . . . n, which engage the proximal most apices of proximal spring 134 and provide longitudinal fixation to the proximal end of stent-graft 130. The number of projections 126 typically corresponds to the number of proximal spring apices. The longitudinal fixation provided by the spindle 122 and its projections 126 provide fixation of the proximal end of the stent-graft to enable its controlled deployment to take place. Sleeve 116 acts as a proximal spring circumferential barrier, and thereby prevents full deployment of the proximal spring. In other words, proximal spring 134 cannot fully deploy until inner tube 120 is advanced to advance tapered tip 112 and sleeve 116 (FIG. 4) to release the proximal crowns.

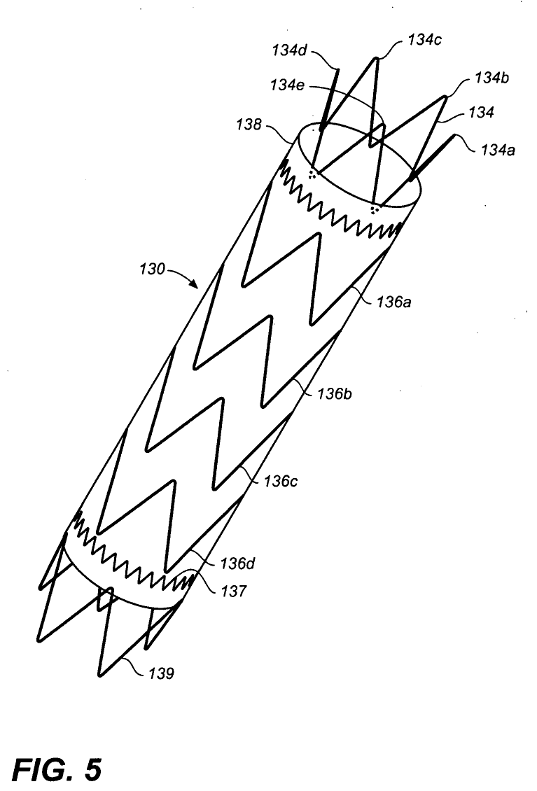

Referring to FIG. 5, stent-graft 130 is shown in a deployed configuration. Stent-graft 130 includes a tubular graft 132, which can comprise polyester or Dacron material and a plurality of annular undulating spring elements or springs that typically are sewn to tubular graft 132 using polyester sutures. These springs, which can be formed from nitinol, include, proximal spring 134, which comprises an annular undulating, annular undulating stents or springs 136a-d, forming a support structure, and an annular undulating sealing spring 138. Proximal spring 134 is attached to tubular graft 132 adjacent to the proximal edge of tubular graft 132. The number of annular spring elements and the number of apices in an annular spring element can vary as would be apparent to one of ordinary skill in the art. In the illustrative example, stent-graft 130 is shown with five proximal spring apices (crowns) 134a-e, optional distal sealing spring 137, and optional bare distal spring 139.

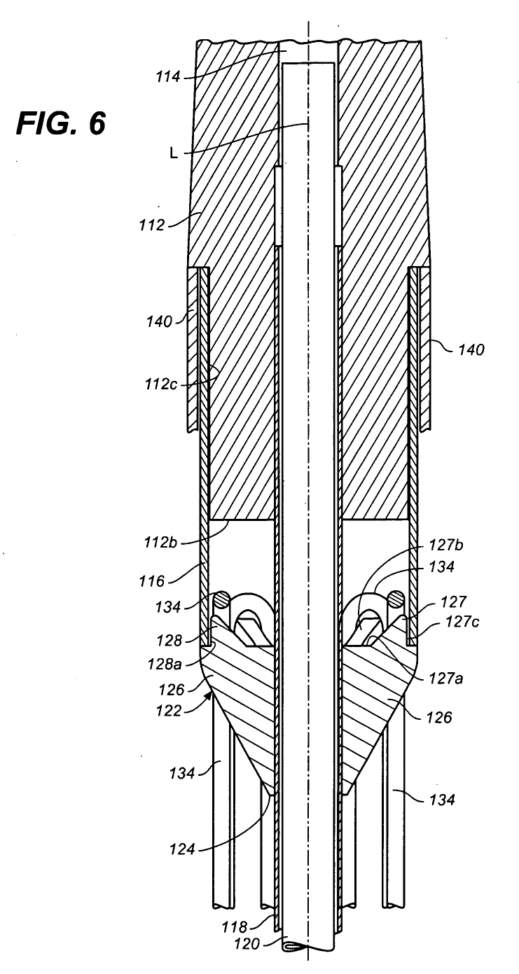

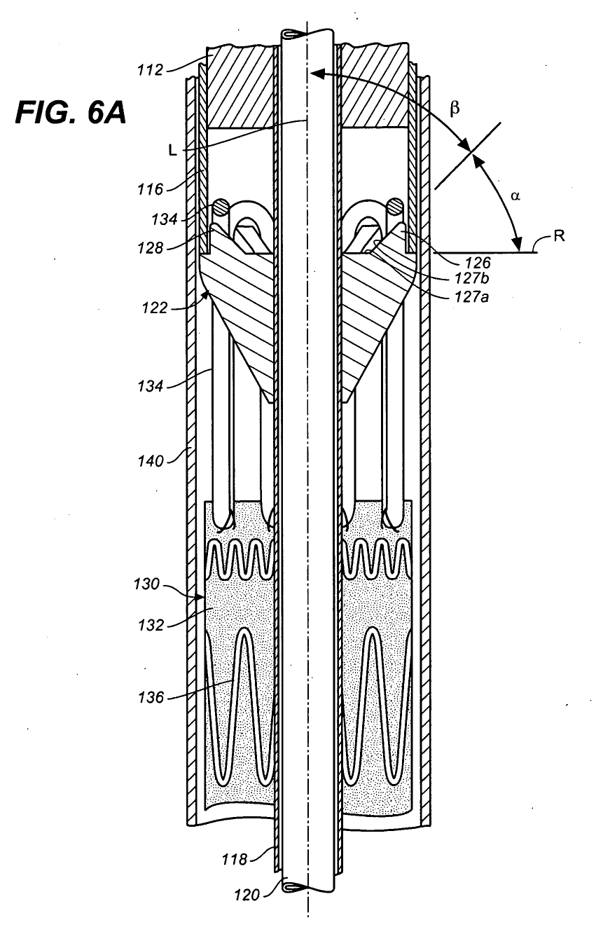

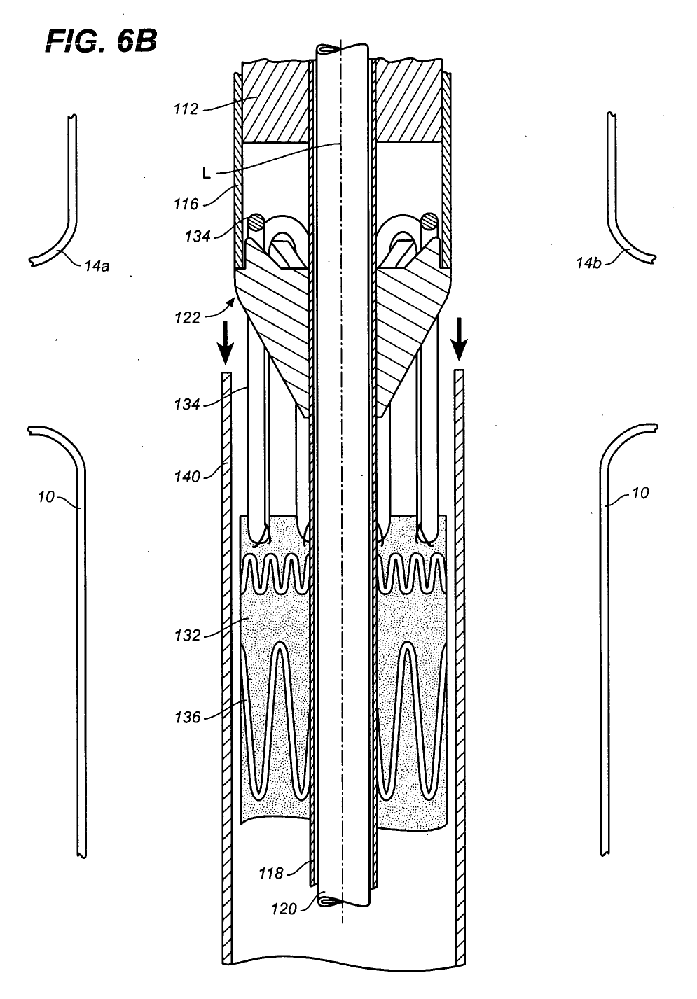

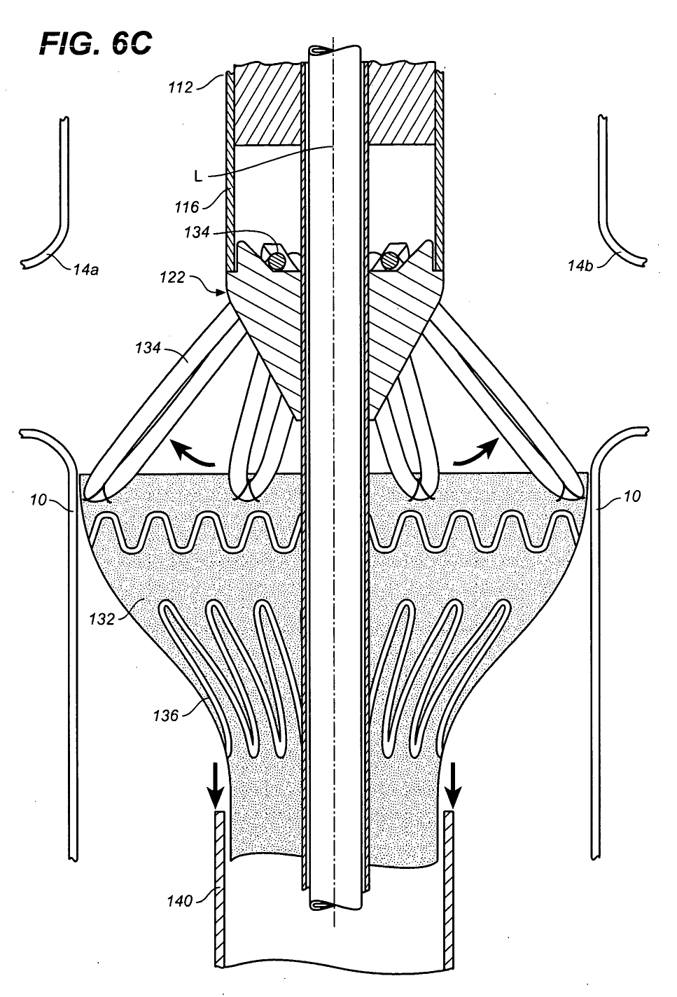

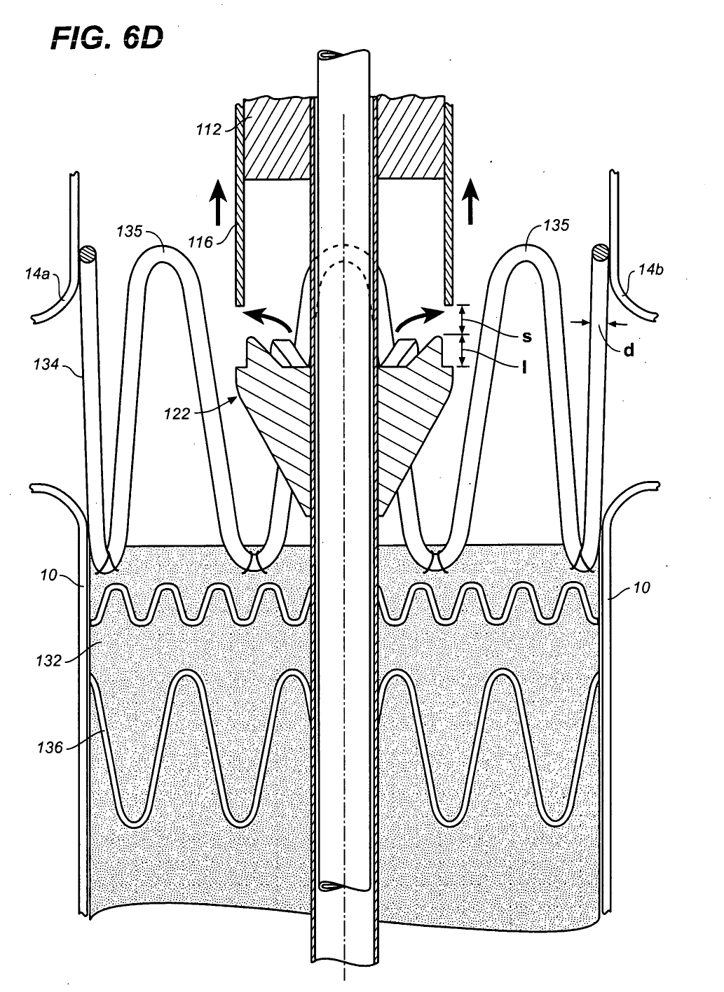

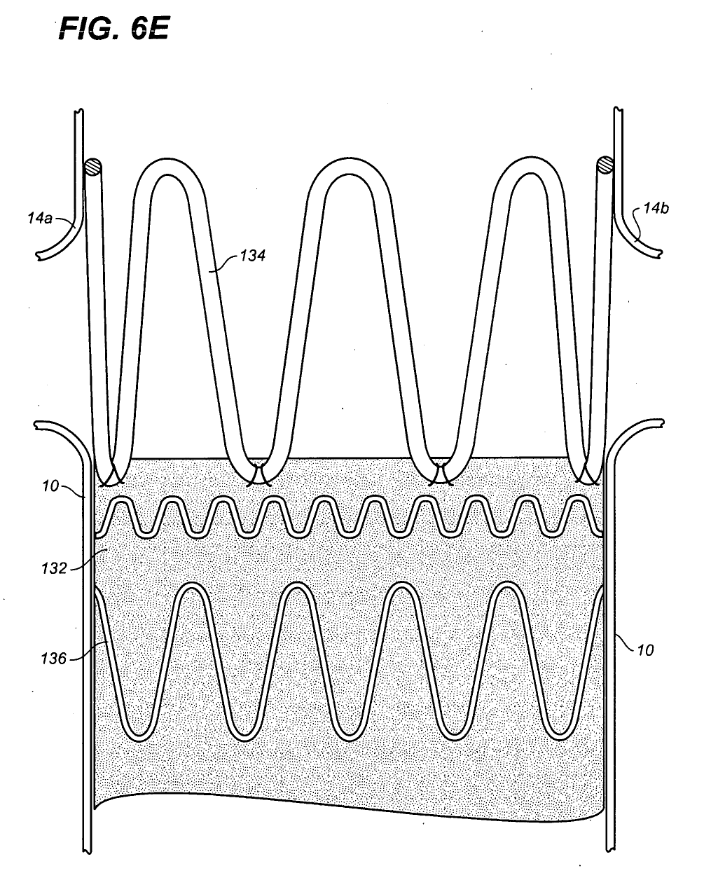

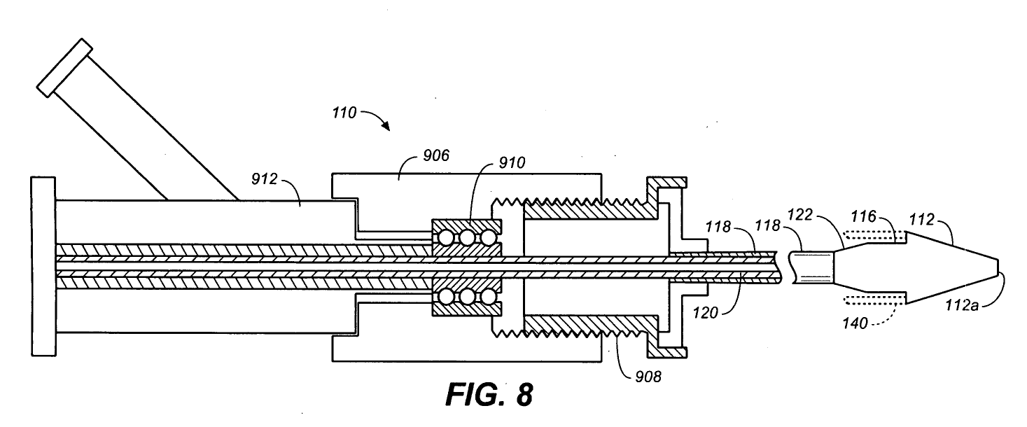

Referring to FIG. 6, an enlarged schematic partial longitudinal sectional view of the distal section of system 110 is shown to illustrate details of the proximal spring proximal crown retention apparatus. Distal tip 112, distal tip lumen 114, and spindle body 124 have a common longitudinal axis “L,” which corresponds to the center line of these elements. Distal tip has a first end 112a (see FIG. 8) and a second end 112b and an annular recess (shoulder) 112c in which tubular sleeve 116 is seated and secured. Spindle body projections 126 extend radially outward from spindle body tubular spindle body 124, which can be integrally formed as a single piece construction with the projections as shown, toward tubular sleeve 116. Projections 126 include teeth 127 extending axially therefrom toward tapered tip. Each tooth 127 includes a first edge face 127a that merges into (connects at a corner to) second edge face 127b. In the illustrative embodiment, the first edge face faces tapered tip 112 and second edge face 127b is angled toward the distal end 112a of tapered tip 112, faces outer tube 118 and forms an angle β of 60 to 30 degrees with first edge face 127a (see FIG. 6A). Additionally, edge face 127b forms a complementary angle a of 30-60 degrees or more typically 45 degrees with a line “R,” which is perpendicular to longitudinal axis “L,” (see FIG. 6A). Consider that an imaginary line extending the second edge face 127b passes through the longitudinal axis “L” at a point along the longitudinal axis to the spindle. The actual angle between the first edge face 127a and the second edge face 127b measured at the corner between the edges is an obtuse angle of 120 to 150 degrees (which is the angle β plus 90 degrees). Each projection 126 includes a recess 128, which includes or is formed by radially extending lip or shelf 128a and tooth edge face 127c that extends parallel to longitudinal axis “L.” Tubular sleeve 116 is seated in the recesses (e.g., 128) and constrains the proximal crowns (tips or apices) of proximal spring 134. Each proximal crown of the proximal spring 134 appears as an upside down U-shaped configuration and straddles a projection and loops or extends over at least one of the first or second edge face.

FIGS. 6A-E illustrate the steps of a method of deploying the stent graft from the distal section shown in FIG. 6. The stent-graft proximal graft edge is placed below the ostia of branch vessels 14a and 14b (e.g., renal arteries) branching from aorta 10. FIG. 6A shows a stent-graft 130 loaded in a stent-graft delivery system (e.g., 110). FIG. 6B shows the delivery system positioned at the desired site adjacent to branch vessels 14a and 14b the stent-graft primary sheath 140 partially retracted. FIG. 6C shows the proximal crowns of the proximal spring pivoting within the constraints of the spindle 122 and the tubular sleeve 116, to allow controlled full radial deployment of the proximal edge of tubular graft 132 of the stent graft. In this configuration, the proximal edge of tubular graft 132 engages the vessel wall (aortic wall 10) and the stent-graft proximal end is laterally centered in vessel 10 prior to release of the proximal spring apices 134a . . . n. In other words, the stent-graft proximal edge is orthogonal to the vessel wall. FIG. 6D shows advancement of distal tip sleeve 116 to release the proximal crowns of the proximal spring apices. The sleeve is advanced a distance “s”, which typically corresponds to twice the length “I” of edge face 127c or twice the cross-sectional diameter “d” of the wire that forms proximal spring 134. The distance “s” can be as little as 2 mm. FIG. 6E shows the stent-graft fully deployed and the delivery apparatus removed.

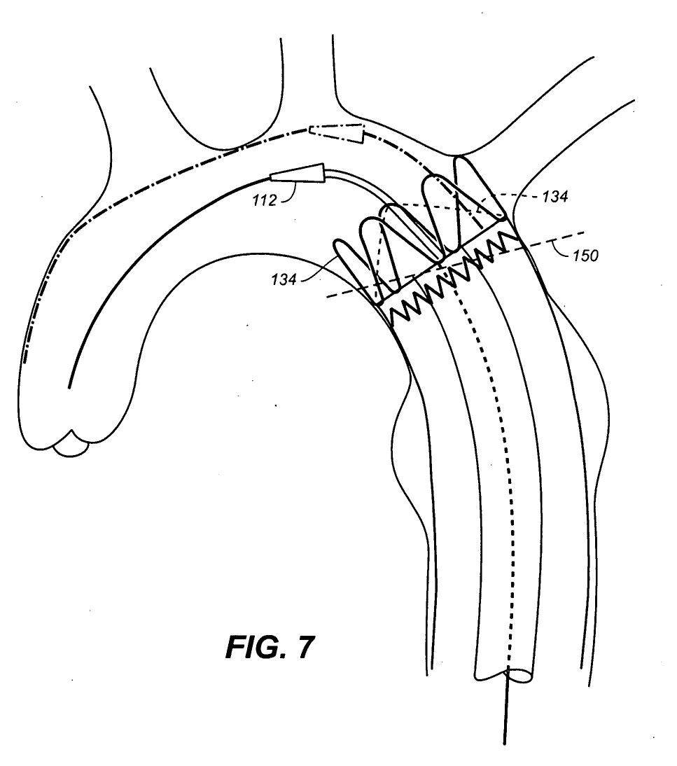

FIG. 7 schematically illustrates deployment of a stent-graft in the ascending aorta using the delivery system of FIG. 1. With the tip release mechanism described above, the proximal edge of tubular graft can be released such that it is deployed along the target line that is orthogonal to the centerline of the aorta as compared to skewed angle line 150 along which it might otherwise be deployed. When using a tip release, which does not allow for the proximal edge of the tubular graft to be positioned against the vessel wall before release of the crown stent apices, the apices when released can release unevenly where some of the apices reach a side wall of the vessel before others and push against a side of the vessel and force the distal tip toward another side of the vessel as shown with dashed lines. This can result in one side of the stent-graft walking back such that the proximal edge of the tubular graft deploys in a non-orthogonal manner, e.g., along line 150, which is offset from the target line.

Any suitable handle having ratcheting mechanisms, threaded mechanisms, or other mechanisms to retract the primary sheath and advance the inner tube relative to the outer tube can be used. Examples of such handles or retraction-advancement mechanisms are disclosed in U.S. Pat. No. 7,264,632 to Wright et al, the disclosure of which is hereby incorporated herein in its entirety by reference thereto U.S. patent application Ser. No. 11/559,754, which published under U.S. Patent Application Publication No. 2008/0114442 on May 15, 2008 to Mitchell et al and is entitled Delivery System For Stent-Graft With Anchoring Pins, the disclosure of which also is hereby incorporated herein in its entirety by reference thereto.

For example, a schematic diagram of a stent-graft deployment delivery system 110 including one of the above-referenced handles and including tip 112 (coupled to an inner tube 120), an outer tube 118 as described herein, as well as a distal assembly or a spinning collar actuation assembly is shown in FIG. 8. The distal assembly provides controlled relative axial movement of the inner tube 120 with respect to the outer tube 118 and preferably includes a luer 912 and spindle 910 attached to the inner tube 120, a threaded shaft 908 coupled to the outer tube 118, and a collar 906. The collar 906 can be attached to the inner tube 118 and yet also spin in relation to the luer 912. In this configuration, the inner tube 120 can advance axially in relation to the outer tube 118 by screwing or spinning the collar 906 down or across the threaded shaft 908. Note that a sheath and stent-graft are not shown in FIG. 8 and the sheath and its actuation mechanism and handle (not shown) are located around the inner and outer tubes as previously described between the stent graft proximal spring apices actuation handle (mechanism) and the tip of the catheter where the stent graft is deployed.

Since the system constrains the proximal crowns of the spring at the proximal end of the stent-graft radially while allowing the middle and/or distal portions of the stent-graft to deploy first, the stent-graft can be repositioned both axially and radially by preventing the stent-graft from fixating itself to a vessel, even when partially deployed. The proximal end of the stent-graft is also axially constrained which enables the delivery system to maintain the position of the stent-graft during the full deployment sequence event if the stent-graft has little or no axial support. Since the present embodiment fixes the proximal end of the stent-graft during deployment while the sheath is withdrawn, the frictional forces between the stent-graft and sheath cause the stent-graft to be held under a tensile load. While under a tensile load, the density of the stent-graft and the compressive forces within the sheath are reduced. Additionally, using the design of the present embodiment, deployment forces can be further reduced by removing supports (such as connecting bars) in the stent-graft since such supports would no longer be needed for deployment.

Any feature described in any one embodiment described herein can be combined with any other feature or features of any of the other embodiments or features described herein. Furthermore, variations and modifications of the devices and methods disclosed herein will be readily apparent to persons skilled in the art.

What is claimed is:

1. A prosthesis delivery system comprising:
a tip having a lumen, a first end and a second end;
a sleeve coupled to said tip second end;
a spindle comprising a spindle body and a plurality of projections extending radially outward from said spindle body, each projection having a first edge face and a second edge face, said first edge face facing said tip and said second edge face extending from said first edge face, being angled toward said tip first end, and extending toward said sleeve; and
endoprosthesis having an undulating spring with a plurality of crown portions having apices, where said crown portions extend over at least one of said first or second edge faces and said sleeve radially constrains said apices.

2. The system of claim 1 wherein each projection has a recessed portion in which said sleeve is seated.

3. The system of claim 1 wherein each first and second edge face of a projection merge and form an angle of 120 to 150 degrees therebetween.

4. The system of claim 1 wherein said spindle body has a longitudinal axis and said second edge face forms an angle of 30-60 degrees with a line perpendicular to and passing through said longitudinal axis, where an imaginary line extending said second edge face passes through said longitudinal axis at a point along said longitudinal axis of said delivery system proximal to said spindle.

5. The system of claim 1 wherein said tip has a tapered outer surface.

6. The system of claim 1 further including a sheath radially constraining said endoprosthesis and releasably coupled to said tip.

7. The system of claim 6 wherein said sheath surrounds said spindle.
